(19) United States
(12) Patent Application Publication
(10) Pub. No.: US 2017/0197075 A1
(43) Pub. Date: Jul. 13, 2017

(54) A TONGUE TREATMENT ELECTRODE AND A DEVICE USING THE SAME

(71) Applicant: KONINKLIJKE PHILIPS N.V., EINDHOVEN (NL)

(72) Inventors: Michel Paul Barbara VAN BRUGGEN, Helmond (NL); Joachim KAHLERT, Aachen (DE); Gary William LOTZ, Mars, PA (US)

(21) Appl. No.: 15/315,060

(22) PCT Filed: May 13, 2015
(86) PCT No.: PCT/EP2015/060682

§ 371 Date: Nov. 30, 2016

Related U.S. Application Data
(60) Provisional application No. 62/009,693, filed on Jun. 9, 2014.

(30) Foreign Application Priority Data
Jul. 10, 2014 (EP) 14176456.3

(51) Int. Cl. A61N 1/05; A61B 17/04; A61B 18/14; A61N 1/36; A61F 5/56

(52) U.S. Cl. CPC A61N 1/0548; A61N 1/3601; A61F 5/566; A61B 18/14; A61B 17/0401; A61B 2018/00577

(57) ABSTRACT

An electrode is provided for use as part of a tongue treatment. The electrode is for providing a permanent anchored implant in the tongue. The anchored implant may be used both for electrical therapy and mechanical (tongue remodeling) therapy.

### FIELD OF THE INVENTION

This invention relates to a tongue treatment electrode for providing electrical therapy to the tongue, and a tongue treatment device using the same.

### BACKGROUND TO THE INVENTION

It is known to use a tongue treatment device to treat upper airway obstruction and sleep disordered breathing.

Respiratory disorders during sleep are recognized as a common problem with significant clinical consequences. Obstructive Sleep Apnoea (OSA) causes an intermittent cessation of airflow. When these obstructive episodes occur, an affected person will transiently arouse. These arousal episodes can occur frequently during the night, so that sleep fragmentation occurs which produces excessive daytime sleepiness. OSA may also lead to cardiovascular and pulmonary disease.

Various approaches are known which aim to maintain the airway passage during sleep.

For example, Continuous Positive Airway Pressure (CPAP) devices are often used as first-line treatments for OSA. These devices use a sealed mask which produces airflow at a slightly elevated pressure and acts to maintain positive air pressure within the airway. This approach requires a mask to be worn, which can cause discomfort to the user. Other treatment approaches have thus been considered, which are aimed at changing the position of the soft palate, jaw or tongue.

An example which aims to control the position of the tongue is known as tongue suspension technology. Tongue suspension devices perform a purely mechanical control of the tongue position. The device comprises a tongue anchor which is surgically applied to the patient. The device advances the tongue in the direction of the anterior mandible, where a bone anchor is mounted, and provides stabilization of the tongue base, so that the tongue can no longer freely move back.

Other approaches are based on electrical treatment of the tongue.

Tongue ablation is a known procedure for removing tongue tissue or reducing the volume of tongue tissue. The tissue is heated to a temperature where it starts to denaturize leading to the formation of stiffer scar tissue with a higher tissue density. This approach can be taken to treat patients with OSA as both these properties have an advantageous effect; stiffer tissue will less readily deform and collapse the airway while higher tissue density leads to a reduction in tissue volume, reducing the probability of tongue collapse.

By way of example, US2009/0149849 discloses a device for modifying the properties of tongue tissue by ablation. Electrodes are connected to power sources located outside of the patient's body. The electrodes are placed either side of the patient's tongue and apply a current to the tongue.

Neuromuscular electrical stimulation (NMES) facilitates reversible and intermittent contraction and relaxation of muscles. This approach provides intramuscular electrical stimulation of the genioglossus muscle to increase the patency of the upper airway.

There are other approaches, such as hypoglossal nerve stimulation (HGNS) or direct nerve stimulation. However these carry greater risks than intramuscular stimulation.

A problem with the different approaches is that they each require a different type of intervention to the patient, so that if multiple treatments are investigated, there can be significant discomfort for the patient.

### SUMMARY OF THE INVENTION

According to the invention there is provided a device as claimed in the independent claim.

Examples of the invention provide an electrode for use in a tongue treatment device for delivering electrical therapy to the tongue tissue, wherein the electrode is for providing a permanent anchored implant in the tongue.

The invention provides an electrode for providing a permanent anchored implant in the tongue and for use in delivering electrical therapy.

This electrode can thus be used for electrical treatment as well as mechanical treatment by its permanent anchoring in the tongue. It can also be used for different electrical treatments while reducing the amount of surgical intervention needed. By permanent implant is meant that the anchor is able to remain in place even after the initial electrical therapy (such as ablation) has been completed. Thus, it can be used for long term electrical treatment and for long term mechanical tongue remodeling.

In one example, the electrode may comprise an implantable expandable anchor which expands from a first tongue insertion configuration to a second deployment configuration, the second deployment configuration being for providing the permanent anchored implant in the tongue.

The electrode may be deployed in a collapsed state, and may expand once deployed into a deployed state for anchoring in the tongue tissue. The implantable tongue electrode is thus easy to implant into the tongue muscle. Once the electrode is deployed it takes the expanded form, so that the implanted electrode provides an anchor in the tongue.

In its expanded form, the electrode provides a permanent anchored implant, so that it is less able to migrate than conventional implants. In the expanded form, a delivery system used to implant the electrode (when it was in its constricted form) is fully retracted, and does not remain in place. Thus, the electrode remains in place after all surgical procedures have been completed. The electrode is thus fully detachable from the tool used to implant the electrode.

In another example, the electrode comprises a conducting loop. This can be implanted using a suture process.

The electrode preferably comprises one or more electrical connection terminals for receiving electrical signals relating to the electrical therapy. In this way, the anchor is used to deliver the received electrical signals to the tongue tissue. The electrode preferably also has a mechanical connection terminal for connection to a mechanical tether line for controlling the tongue position. In this way, the anchor is used as for mechanical control of the tongue position or movement, for example to limit the tongue movement. The electrical connection terminals and the mechanical connection terminals may be the same or different,

The invention also provides a tongue treatment device comprising:

an electrode as described above;

an electronic driver arrangement;

an electrical supply line provided between the electronic driver arrangement and the electrode for delivering electrical power to the electrode; and

a bone anchor for attachment to the anterior mandible, wherein the electrical supply line comprises a mechanical tether line which couples the electrode to the bone anchor or else a further connection line is provided which couples the electrode to the bone anchor.

In use of the tongue treatment device, the electrode is implanted into a patient's tongue and the electronic driver arrangement may power the electrode via the electrical supply line such that the electrode transfers electrical energy to surrounding tongue tissue.

By way of example, the anchored electrode may in some embodiments be used as a tissue anchor in a tongue remodeling system. The additional ability to supply electrical energy to the electrode means the electrode can be used to provide not only tongue remodeling, but also treatments which involve delivering electrical energy to the tongue tissue, such as intramuscular stimulation and/or ablation. These involve applying electrical therapy to the tongue tissue.

For example, the ablation of tongue tissue can result in a more secure fixation of the electrode for subsequent use as part of a tongue remodeling system, or indeed as part of a tongue muscle stimulation system.

The bone anchor may be fixed to a patient's anterior mandible by screws. The bone anchor can be used as part of a tongue remodeling system and it can be used to mount the electronic driver arrangement.

The electronic driver arrangement of the tongue treatment device may thus be at, e.g. attached to, the bone anchor. The driver may then provide power to the electrode for electrically stimulating the tongue. The driver may thus be inside the patient's body and can therefore be used by the patient without causing the discomfort associated with a mask or other external equipment.

In one application, the electronic driver arrangement is adapted to provide the electrode with signals suitable for carrying out ablation. For example, for resistive ablation, the electronic driver arrangement may supply power to the electrode to deliver pulses of energy between 10 W to 50 W, at a frequency of 100 kHz to 500 kHz. The electronic driver arrangement may be adapted to power the electrode so that the electrode delivers pulses having a duration of between 1×10⁻³ s and 1 s. To carry out the ablation treatment, the electronic driver may drive the electrode to deliver between 1 and 10 pulses per treatment. These signals are suitable for ablating tongue tissue. The amount of deposited energy will determine the temperature of the tissue and the size of the treated tissue. It may be preferable to heat the tissue to a temperature which guarantees apoptosis (cell death) of the surrounding tissue while avoiding overheating of the tissue. For example, heating to 80° C. may be appropriate.

The electronic driver arrangement may instead be adapted to provide signals suitable for dielectric ablation, for example at a frequency greater than 10 MHz.

In another application, the electronic driver arrangement is adapted to provide the electrode with signals suitable for carrying out intra muscular stimulation. For example, the electronic driver arrangement may supply the electrode with a current of between 1 to 100 mA, and power the electrode to deliver pulses of power having a frequency of between 1 to 25 Hz and a pulse duration of 1×10⁻⁶ s to 1×10⁻³ s. These signals are suitable for stimulating the tongue to cause contraction and relaxation. The stimulation system can for example be used by a patient during sleep.

When the driver is for carrying out tongue ablation, it may be external to the patient's body (rather than attached to a bone anchor). In particular, the tongue ablation treatment is typically a once-only procedure carried out under general anaesthetic.

Thus, the electronic driver arrangement can comprise two separate units—one for providing stimulation signals and for mounting at the bone anchor and one for providing ablation signals from an external (higher power) driver. However, it is also possible for a single driver unit to provide both types of signal.

The electronic driver arrangement may then be configurable to operate in a first mode in which the tongue treatment device is adapted to provide tongue tissue ablation, and a second mode in which the tongue treatment device is adapted to provide intra muscular stimulation.

There is thus provided an overall system which may implement both tongue tissue ablation and intra muscular stimulation. The same tongue anchor electrode can be used for each treatment. In this way, the amount of surgical intervention required is reduced to enable ablation and/or tongue stimulation therapy to be provided. In this way, the device may provide either permanent or temporary advancement of the tongue, or a combination of both, in an approach that may be both technically simple and cost effective. The ability to provide both treatments of ablation and stimulation using a single device is advantageous, as the electrode can be used to initially treat the tongue using ablation to change the properties of the tongue, and create a better environment for the tongue anchor to carry out stimulation treatment.

As explained above, a bone anchor may be used for mounting the electronic driver, particularly for muscle stimulation treatment.

The bone anchor is additionally used as part of a tongue remodeling system. For this purpose, a mechanical tether line is provided. The electrical supply line may itself function as the mechanical tether line which couples the tongue anchor electrode to a bone anchor. Alternatively, a separate mechanical tether line may be provided. The tether line is then used to restrict movement of the tongue to prevent the tongue from blocking the airway. In this way, the electrode can be used as the tongue anchor of a physical tongue manipulation device.

This type of treatment is known, for example from US2008/0023012.

As mentioned above, the line that supplies electric power to the implanted electrode can itself be used as the mechanical tether line. Instead, the electric line may be released from the tongue anchor after the electric treatment (ablation and/or stimulation) is complete, and a mechanical cable can then be fixed to the tongue anchor, which cable forms part of a tongue manipulation device.

The tongue remodeling system may comprise a spool part at the bone anchor for spooling of the tether line to advance the tongue in the direction of the mandible. In addition, the bone anchor may comprise a locking feature to lock the bone anchor after tether spooling is complete.

This provides the user an advantage in that a patient must undergo just one surgical procedure to fit the device (bone anchor and tongue anchor), and it is then possible to use the device to implement different possible methods of treating the tongue without further surgical intervention.

Of particular interest is the use of a shared tongue anchor electrode for remodeling and stimulation. This provides two possible treatments using the same system. The possibility of tongue ablation and stimulation is also of interest, in that the tongue ablation causes improved fixation of the electrode in the tongue.

The system may however be used for stimulation only.

The tongue treatment device may comprise a plurality of electrodes, to provide a multi-point device. In this way, it is also possible to smoothly heat a large volume of tissue for ablation, rather than heating a small area, as is the case with conventional needle like RF ablators.

The tongue anchor electrode may comprise a sensor for sensing muscle activity, such as a pressure sensor. The sensor may provide the electronic driver arrangement with information indicating whether an OSA event is likely to take place. The electronic driver arrangement may then generate stimulation signals in response. This provides a feedback signal to enable the treatment (in particular muscle stimulation) to be carried out when required.

The pressure sensor may act to measure position such that if the posterior pressure is determined to increase, or is greater than a threshold value, electrical stimulation is triggered.

The electrode may be a super-elastic metal alloy. This may allow for convenient delivery of the electrode. For example, the electrode may be folded up such that the electrode can be inserted into a delivery tube of small diameter.

The electrode of the tongue treatment device may be a conducting loop of tether material. For example, the loop may be of conductive textile material. The loop may be designed to have a conductive exposed portion of the loop to which the tongue tissue is exposed, and the rest of the loop is insulated.

An embodiment of the invention also provides a tongue treatment method comprising:

implanting an electrode into the tongue to provide a permanent anchored implant in the tongue; and

providing treatment signals to the electrode from an electronic driver arrangement along an electrical supply line provided between the electronic driver arrangement and the electrode.

The electrode implantation may comprise:

implanting the electrode into the tongue in a first tongue insertion configuration using an implantation tool;

expanding the electrode to a second deployment configuration, thereby to provide the permanent anchored implant in the tongue; and

removing the implantation tool to leave the expanded electrode in the tongue.

### BRIEF DESCRIPTION OF THE DRAWINGS

Examples of the invention will now be described in detail with reference to the accompanying drawings, in which:

FIG. 1 shows a known tongue treatment device for example as disclosed in US 2009/0149849.

FIG. 2 shows a first example of a tongue treatment device;

FIG. 3 shows how the device can be designed to provide mechanical tongue manipulation;

FIG. 4 shows a second example of a tongue treatment device;

FIG. 5 shows various different treatment combinations that can be implemented using the system; and

FIG. 6 shows in cross section three possible electrical supply line designs.

### DETAILED DESCRIPTION OF THE DRAWINGS

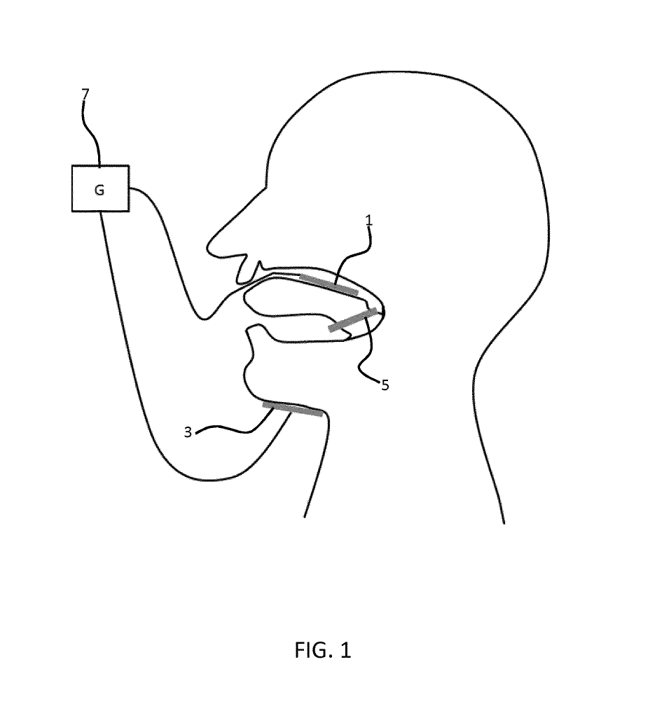

Various tongue treatment devices for treating OSA are known, as described above. By way of example, FIG. 1 shows a known treatment device for providing tongue ablation, in use.

A monopolar first electrode 1 is disposed on the tongue and a ground (return) electrode 3 contacts the patient's skin under the chin. An alternative bipolar electrode version has two tongue electrodes opposite each other so that the tongue is between the two electrodes. A section of the tongue is deformed by an organ shaping device 5 which is located between the electrodes. An electric current is applied to modify muscle tissue according to the current density gradient, which is provided by the surface areas of the electrodes and the deformed section of the tongue. The electrodes receive power from a generator 7 located externally to the patient's body.

It has also been proposed to provide a temporary implanted tongue electrode for tongue ablation.

The invention provides an electrode for a tongue treatment device, and the tongue treatment device itself. The electrode is for providing a permanent anchored implant in the tongue. The electrode is then used for electrical treatment, but it can also be used for other purposes as explained below. The electrical treatment may comprise tongue ablation in the same general manner as the device of FIG. 1, but it may additionally or alternatively comprise tongue stimulation. Instead of providing electrodes on the tongue surface, or a temporary implanted electrode, a permanent implanted tongue electrode is provided which thus performs both a mechanical fixing function as well as a function of providing an electrode for use in ablation and/or stimulation treatment.

An electronic driver arrangement maybe arranged for powering the tongue anchor electrode, and an electrical supply line is provided between the electronic driver arrangement and the electrode.

The permanent implantation of the electrode means the electrode is less likely to migrate once it has been implanted into the tongue. Therefore, the electrode can be used both to deliver electrical energy to tissue and to provide an anchor in the tongue tissue, for example for treatment based on a mechanical tongue suspension.

The treatment device of the invention may be designed for different possible treatments. A first example is the combination of muscle stimulation and mechanical tongue remodeling. Other examples comprise stimulation only, stimulation and ablation and ablation only.

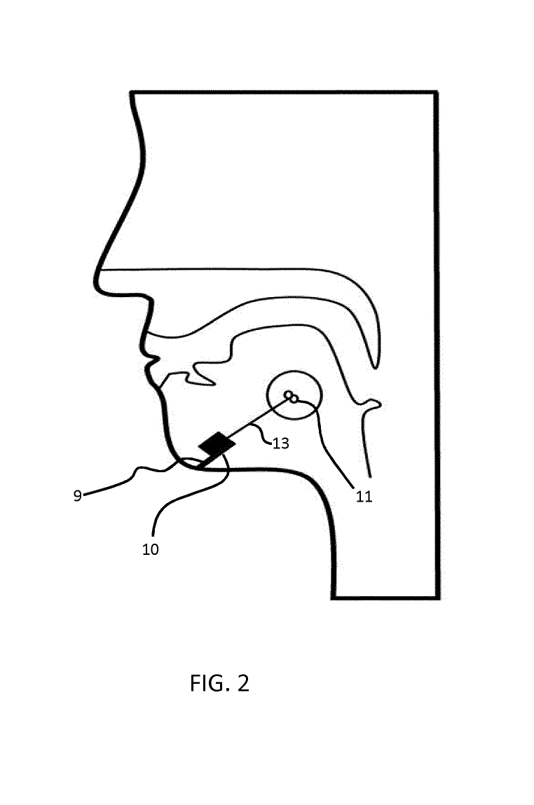

FIG. 2 shows a first example of a treatment device in accordance with the invention for providing the combination of muscle stimulation and mechanical tongue remodeling. The device comprises a bone anchor 9 which is attached to the surface of the mandible and an implanted electrode 11 which functions as a mechanical anchor.

The bone anchor 9 carries a drive circuit 10 with its own battery power supply, and the drive circuit is coupled to the electrode 11 by an electrical supply line 13.

The battery stores enough energy to power the device over consecutive nights. The drive circuit is switchable to turn the device on and off remotely. The electronic driver 10 comprises a signal generator, and a clock to allow a user to time the onset and end of treatment. This may be of interest for treatment taking place overnight, for example.

Further, the electronic driver comprises a controller for controlling the signal generator and a memory to store and change stimulation patterns. In an example, the memory is a random-access solid state memory.

The bone anchor 9 is connected by bone screws to the mandible bone, and the electrode 11 is embedded in the tongue. In one example, the electrode 11 comprises an implantable expandable tongue anchor, which is implanted into the tongue, to deliver electrical energy to the surrounding tongue tissue in response to a signal. The electrode may be a mono-polar or a bipolar radiofrequency (RF) electrode.

The procedure for implanting this type of electrode involves inserting the electrode into the ventral side of the tongue. The electrode 11 is folded in an electrically isolated delivery tube and inserted from under the chin, using a trocar to locate a target area of the tongue where the electrode should be inserted. Once the correct position in the tongue base is located, the folded electrode 11 is deployed. In an example, the electrode 11 is of super-elastic metal alloy to allow for convenient insertion into the tongue. The electrode 11 can be folded into the electrode delivery tube which has a diameter of only a few millimeters, and deployed so that the electrode is deposited into the tongue tissue, where it expands to provide a permanent anchored implant in the tongue for use as a tongue anchor in a mechanical tongue suspension treatment.

In this example, the tongue treatment device is used to provide a combination of mechanical tongue suspension and stimulation treatment, making use of the same tongue anchor/electrode. The electrical supply line 13 providing energy to the implanted electrode thus functions as a mechanical tether line. The bone anchor then includes a spool arrangement, enabling a surgeon to spool the tether line into the bone anchor. This process is called titration or adjustment and stabilizes the tongue as well as advances the tongue in the direction of the anterior mandible (or prevents the tongue moving back), preventing blocking of the airway. The spool arrangement comprises an indexing part and a spool part, so that a tangible as well as an audible sound is made during titration so that the degree of tightening can be judged. The indexing part can also retain the spool in a fixed position after adjustment or a separate lock can be used.

The tether line 13 then comprises an isolated conductive structure which electrically and mechanically connects the tongue anchor and bone anchor.

Following insertion, the tongue anchor electrode 11 is used for tongue stimulation, using the bone anchor 9 and driver 10 as shown in FIG. 2.

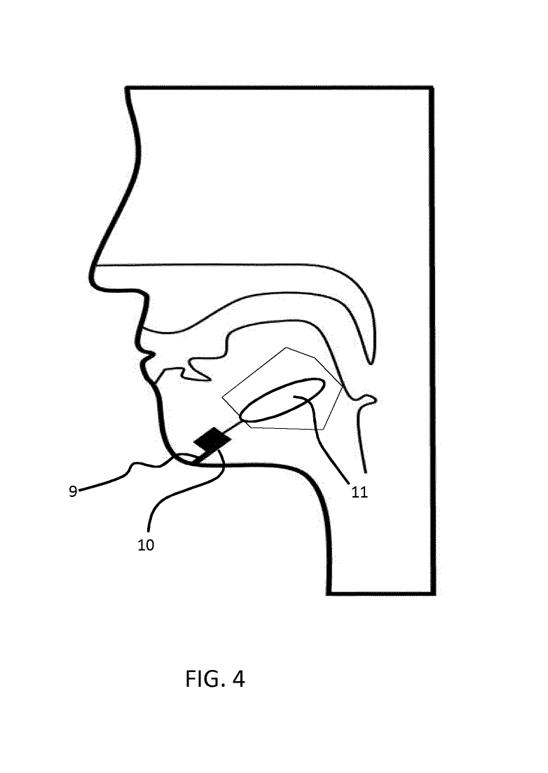

Generally, the implant is placed in the midline of the tongue at the base of the tongue and the device provides stabilization and advancement of the tongue base, so that the tongue can no longer move freely back. Instead, it is blocked by the tether line between the bone and tissue anchor. The tissue area, which is the target zone for the implant, moves by physiological events such as swallowing, yawning and speech; up and down and forwards and backwards. The electrode 11 can be formed as a barb type structure as discussed above or as a loop of the conductive material as shown in FIG. 4.

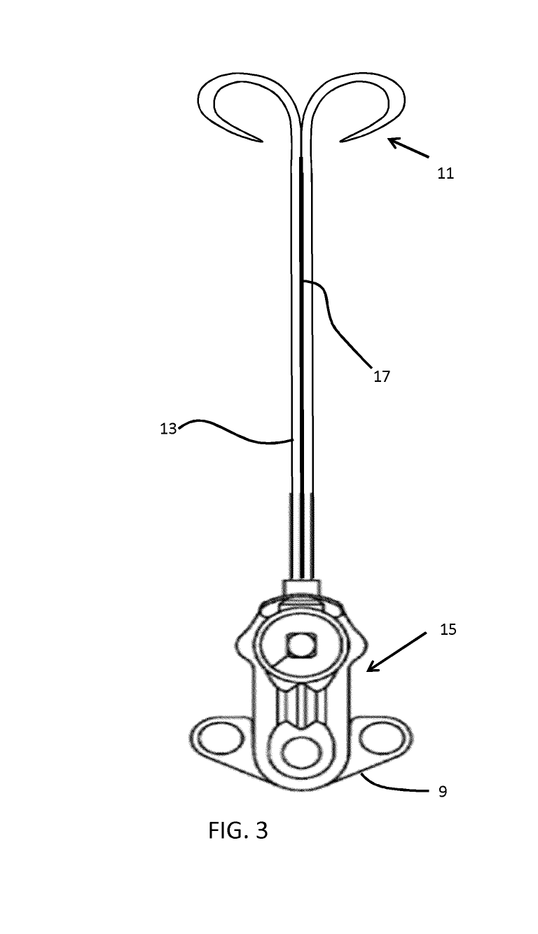

FIG. 3 shows more clearly the features which provide the mechanical tongue remodeling. The bone anchor 9 comprises a tether line spool arrangement 15 which enables the tether line 13 to be wound onto the spool to take up slack in the tether line 13 to provide a desired restricted movement of the tongue. The controller additionally connects electrically with the tether line, so that the tether line 13 has a conducting core 17 which provides electrical supply between the controller 10 at the bone anchor 9 and the electrode 11.

The tongue anchor electrode 11 in this example comprises a set of barbs, which when deployed face towards the direction of insertion of the tongue anchor into the tongue tissue. In this way, they provide a permanent anchoring function, in the sense that removal of the tongue anchor from the tongue requires a further medical intervention, and the tongue anchor is able to remain in place for prolonged periods (months or years) and designed to prevent migration out of the tongue tissue. The tongue anchor can of course be removed by a surgical procedure, which involves retracting the expandable barbs back into a retraction tube for removal from the tongue.

The tether line 13 delivers electrical energy to the tongue tissue in order to stimulate the tongue, and further restricts mechanical movement of the tongue. In this way, a user can undergo only one implant procedure, and the system is then able to provide both treatments.

FIG. 4 shows that for providing intramuscular stimulation, the inserted electrode can be formed as a conducting loop. Such a conducting loop can also function as a permanent anchor, and indeed a permanent anchor in the form of a loop for use in a tongue remodeling system is for example known from WO 2011/123714. This system is also commercially realized, as the Encore (trade mark) system of Siesta Medical (trade mark).

The loop is implanted using a suture passer system.

A conductive area of the loop can be exposed, for transferring electrical energy to the tongue tissue. The rest of the loop (where it passes between the bone anchor at which the driver is located and the tongue tissue) is isolated by non-conductive material.

As for the barb type anchor described above, this form of loop anchor can again provide the multiple functions of tongue anchoring in a tongue remodeling system, and providing electrical impulse signals.

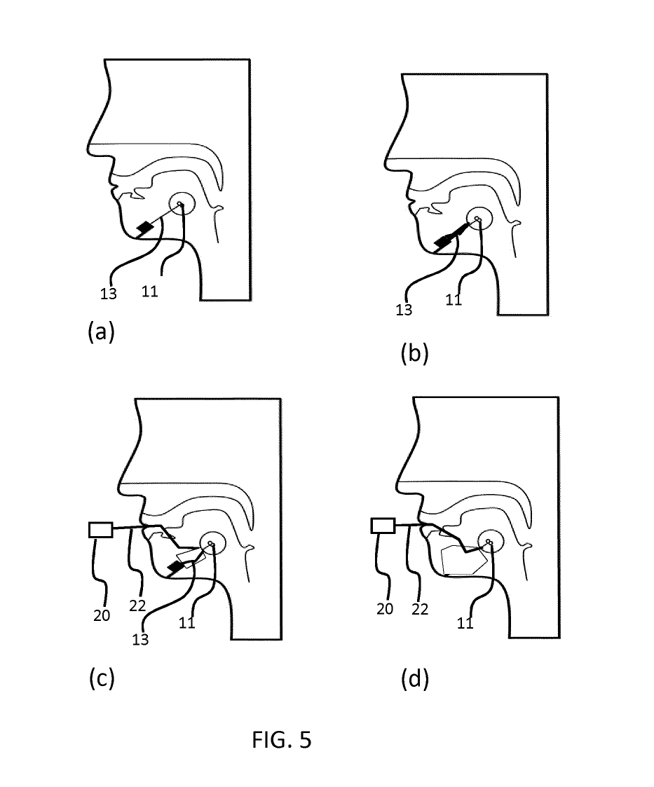

The example above is based on the combination of tongue remodeling and muscle stimulation. However, the implanted electrode anchor may be used for other treatments and combinations of treatments, some examples of which are shown in FIG. 5.

FIG. 5(a) shows the combination of tongue remodeling and stimulation as described above. The electrical line 13 functions as both a taught tether line and an electrical supply line, between the bone anchor and the tongue. The electrode 11 can be formed as a loop of the conductive material as shown in FIG. 4 or a barb type structure.

FIG. 5(b) shows a system for tongue stimulation only. In this case, it is not necessary for the bone anchor to comprise a spool part. The supply line 13 does not need to provide a mechanical support function, and is schematically shown as slack. The electrode 11 can be formed as a loop of the conductive material as shown in FIG. 4 or a barb type structure.

In use, electrical energy is applied to the tissue surrounding the electrode 11 in order to achieve stimulation of the tongue. For the stimulation procedure, the driver 10 supplies the tongue anchor electrode 11 with power through the electrical supply line 13. The electrode 11 can be used to electrically stimulate the tongue, causing the muscle to perform a contracting action (intramuscular stimulation), which results in temporary opening of the upper airway. The level of contraction and the level of anterior advancement of the tongue resulting from stimulation are determined by the amount stimulation energy delivered to the tongue. Stimulation may also be used to treat patients who have had injury to the genioglossus, for stroke and support treatment, dysphagia and dysarthria.

For muscle stimulation, a current of between 1 to 100 mA is for example appropriate, with pulses having a frequency of between 1 to 25 Hz and a pulse duration of 1×10⁻⁶ s to 1×10⁻³ s.

FIG. 5(c) shows a system for tongue stimulation and ablation. In this example, a separate driver 20 is provided for the ablation treatment. It has an electrical supply line 22 which connects to the tongue anchor electrode. After the ablation is complete, the driver is removed, and connection is made between the driver at the bone anchor and the tongue anchor electrode.

Again it is not necessary for the bone anchor to comprise a spool part. The supply line 13 does not need to provide a mechanical support function, and is schematically shown as slack. The electrode 11 can again be formed as a loop of the conductive material as shown in FIG. 4 or a barb type structure.

For muscle ablation, the electronic driver arrangement may deliver energy as pulses with power 10 W to 50 W to the tongue tissue for example at a frequency of 100 kHz to 500 kHz. The pulses may have a duration of between 1×10⁻³ s and is and between 1 and 10 pulses per treatment may be appropriate. The electrical supply leads for ablation must be suitable for carrying at least the ablation electrical power load. In contrast, for a device which is used only for stimulation the power leads may have less resistance as Ohmic heating is not required.

In the example of FIG. 5(c), there are separate drivers for tongue ablation and tongue stimulation, and these together may be considered to be an electronic driver arrangement. The different drivers could instead be part of a single device at the bone anchor.

In either case, the electronic driver arrangement is configurable to operate in a first mode in which the tongue treatment device is adapted to provide tongue tissue ablation, and a second mode in which the tongue treatment device is adapted to provide intra muscular stimulation. The modes may comprise use of a selected driver, or they may comprise the operation of a single driver in a suitable manner. In either case, the treatment device enables different treatments to be selected, but making use of the same implantable tongue anchor electrode 11.

All three treatments may be provided by combining the external ablation driver 20 with the stimulation and remodeling version shown in FIG. 5(a).

FIG. 5(d) shows a system for ablation only. The electrode 11 can be formed as a loop of the conductive material as shown in FIG. 4 or a barb type structure.

In embodiments making use of muscle stimulation, the tongue anchor electrode 11 may comprise a sensor for sensing muscle activity. The sensor senses low voltages generated by the muscle fibres in the tongue to provide an electromyogram of the genioglossus muscle. The electromyogram correlates to the muscle activity of the tongue and provides information as to the stiffness and advancement of the genioglossus muscle. This information may be used to determine how treatment is carried out.

The sensor may be a pressure sensor and, for example, indicates tongue position by signaling whether posterior pressure is increasing or is greater than a threshold value. The controller 10 of the bone anchor processes the signals from the sensor and, based on the received information, an electrical signal generator creates an electrical stimulation signal. If the sensor signals that an OSA event is likely to occur, the signal generator creates an electrical stimulation signal which is transmitted by the electrical supply line to the tongue anchor electrode. The electrode then delivers electrical energy to the surrounding tongue tissue in order to prevent signaled OSA event from occurring.

In other embodiments, the sensing may be achieved by an alternative means such as breathing/flow sensing, respiratory drive sensing, oxygenation sensing or sensing of snoring etc.

In another embodiment also making use of tongue stimulation, the device stimulates the tongue in response to a remote control. In such an embodiment, the remote control sends a signal to the device instructing the device to start or stop treatment. The device may be pre-programmed to deliver treatment at a certain time, or after a specified time delay. For example, the device may be pre-programmed to begin stimulation after a time delay which reflects a typical duration of time taken for a patient to fall asleep, such that the onset of stimulation occurs when a patient is sleeping.

For examples making use of tongue ablation, electrical current is applied to the electrode 11, and flows through the electrode 11 to the tongue tissue to ablate the tongue. If the electrode 11 is a monopolar electrode, a reference electrode may be attached to the body at another location (as shown in the example of FIG. 1). For a bipolar electrode, the current flows through the electrode then back into the ablation driver system.

Locally heating the tongue tissue surrounding the electrode leads to tissue scarring and tissue shrinkage. Reducing the volume of the genioglossus muscle leads to a reduced probability of tongue collapse and can result in permanent opening of the upper airway at the level of the base of the tongue. A further advantage of forming scar tissue is that scar tissue provides a more suitable environment for the implant. Scar tissue is a better environment for the tongue anchor to anchor in as it is stiffer and provides better mechanical properties. Further, the tongue anchor is less likely to migrate from the tongue when surrounded by scar tissue compared to normal tissue. Additionally, inflammation after permanent and dynamical mechanical stress is less likely in scar tissue than in normal tissue. It may therefore be preferable to ablate the tongue tissue soon after insertion of the electrode, in order to provide a better environment for the electrode to be anchored in the tongue where it may subsequently carry out intra muscular stimulation and/or be used as part of a mechanical tongue remodeling system.

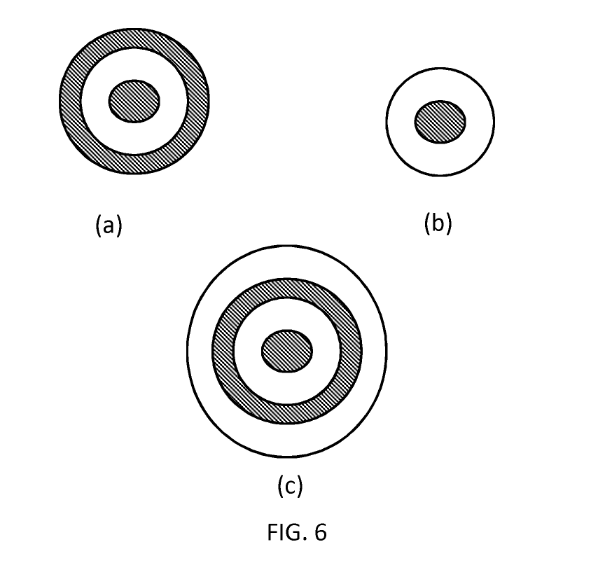

Various designs for the electrical supply line are possible. FIG. 6(a) shows in cross section a concentric arrangement, with a conductive inner core and a conductive outer sheath separated by an insulation layer. The outer sheath functions as the counter electrode, and the stimulation or ablation feed is provided along the central core. The counter electrode is earthed by the driver circuit.

FIG. 6(b) shows a monopole version which comprises a central core surrounded by insulation. Where implanted, the outer insulation will be omitted. FIG. 6(c) shows that two supply lines can connect to the tongue anchor electrode which are insulated externally.

The power profile of the electrical signal will differ for stimulation and ablation. Ablation will use higher power and longer duration pulses, but repeated at a lower frequency. The power delivered will influence the depth to which ablation takes place, for example based on:

P×Δt=c×ΔT×A_{elec}×d_{ablation}

P is the power applied, Δt is the pulse duration, c is the heat capacity of the tongue, A_{elec} is the electrode surface area and d_{ablation} is the ablation depth.

The ablation depth may be selected for different purposes. For example a shallow ablation depth can be used to provide a thin layer of scar tissue to prevent microbleeding. A thicker ablation depth can be used change the macroscopic tissue properties to give a higher stiffness and density (as a result of a reduced volume).

The electrode/anchor design described above has backward facing barbs, which curl out from a delivery duct. Instead, the anchor may comprise projections which extend radially when deployed, for example metal tines which are deployed from slits in the outer side wall of the delivery duct instead of from an opening at the end. Thus, different types of expandable anchor may be used. Essentially, the anchoring is provided by inserting expanding features in a direction which does not correspond with the direction of insertion into the tongue, so that there is tongue tissue preventing the withdrawal of the anchor along the same path that it was inserted before expansion. The sutured based version described above is a further alternative.

As explained above, there are different possible treatments.

For ablation, there are also different options.

Resistive ablation involves applying a potential difference between the tongue tissue and ground. This can be provided at radio frequencies. This requires two electrodes at different potentials.

Dielectric ablation at microwave frequencies can use a monopolar electrode, without the need for a separate ground electrode. This requires only one electrode, which functions as a microwave antenna.

A monopolar supply line (FIG. 6(b)), can be used straightforwardly for dielectric ablation since only one electrode is needed. It can however also be used for resistive ablation. In this case, a grounded counter electrode can be placed at one or more positions on the patient, such as under the chin, on the back of the neck or on top of the tongue.

An alternative way to provide the two electrodes for bipolar resistive ablation is to form different tines of the implanted tongue electrode as separate isolated electrodes. There can then be two supply lines to the tongue anchor electrodes (e.g. using a supply line as in FIG. 6(c)).

The ablation can be carried out by supplying energy using the implantation tool so that the ablation process is part of the surgical procedure before the tongue anchor electrode is left in place. For example, the implant tool may provide a pair of isolated supply lines to the tongue anchor, with a first set of anchor tines connecting to one supply line and a second set of anchor tines connecting to the other supply line.

Alternatively, the ablation can be carried out after the surgical procedure is complete by supplying the ablation energy from the driver at the bone anchor. The driver may not have the required power for ablation, and the ablation energy can for example be routed from an external supply to the supply line, to then feed energy to the tongue anchor electrode. The ablation procedure can then be separate to the surgical implant procedure. Again, this can be with two separate supplies to two sets of the tongue anchor tines, or else using temporary ground electrodes. Dielectric ablation can be carried out using energy supplied externally to the supply line at the bone anchor end.

The outside of the implant tool may itself function as a ground electrode.

Thus, it will be seen that there are various options for using the implant tool as part of the ablation treatment or using the driver. There are also various possible designs for the tongue anchor electrode, either as a single monolithic metal component with all tines carrying the same voltage or as a structure with electrodes in two sets so the voltage can be applied between them, or as a loop structure.

The stimulation treatment is provided by the driver at the bone anchor.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measured cannot be used to advantage. Any reference signs in the claims should not be construed as limiting the scope.

1. (canceled)

2. The device as claimed in claim 1, wherein the electrode comprises an implantable expandable anchor which is adapted to expand from a first tongue insertion configuration to a second deployment configuration, the second deployment configuration being for providing the permanent anchored implant in the tongue.

3. The device as claimed in claim 1, wherein the electrode comprises a conducting loop.

4. The device as claimed in claim 1, wherein the electrode comprises one or more electrical connection terminals for receiving electrical signals relating to the electrical therapy.

5. The device as claimed in claim 1, wherein the electrode comprises a mechanical connection terminal for connection to the mechanical tether line for controlling the tongue position.

6. The device as claimed in claim 1, wherein the electrode comprises a plurality of electrode portions.

7. (canceled)

8. The device as claimed in claim 7, wherein at least a part of the electronic driver arrangement for providing intra muscular stimulation is at the bone anchor.

9. The device as claimed in claim 8, wherein the electrical supply line comprises an isolated conductive structure for providing electrical supply between the electronic driver arrangement and the electrode as well as a mechanical coupling.

10. The device as claimed in claim 8 comprising a spool at the bone anchor for spooling the tether line.

11. (canceled)

12. The device as claimed in claim 7, wherein the electronic driver arrangement is adapted to provide signals for one or more of:
monopolar resistive tongue ablation;
bipolar resistive tongue ablation;
single electrode dielectric tongue ablation.

13. The device as claimed in claim 7, wherein the electrode comprises a sensor for sensing muscle activity such as a pressure sensor.

14. (canceled)

15. The device as claimed in claim 13 wherein the electronic driver arrangement is adapted to provide electrical stimulation signals to the electrode in response to signals received from the sensor.

16. A tongue treatment device comprising:
an electrode for providing a permanent anchored implant in the tongue for delivering electrical therapy to the tongue tissue, wherein the electrode is for providing a permanent anchored implant in the tongue;
an electronic driver arrangement;
an electrical supply line provided between the electronic driver arrangement and the electrode for delivering electrical power to the electrode; and
a bone anchor for attachment to the anterior mandible, wherein the electrical supply line comprises a mechanical tether line which couples the electrode to the bone anchor or else a further connection line is provided which couples the electrode to the bone anchor,
wherein the electronic driver arrangement is configurable to operate in a first mode in which the tongue treatment device is adapted to provide tongue tissue ablation, and a second mode in which the tongue treatment device is adapted to provide intra muscular stimulation.

17. An electrode for use in a tongue treatment device for delivering electrical therapy to the tongue tissue, wherein the electrode is for providing a permanent implant in the tongue and for providing an anchor for connection to a mechanical tether line.

18. An electrode as claimed in claim 13, comprising an implantable expandable anchor which is adapted to expand from a first tongue insertion configuration to a second deployment configuration, the second deployment configuration being for providing the permanent anchored implant in the tongue.

19. An electrode as claimed in claim 13, wherein the electrode comprises a conducting loop.
